(19) United States
(12) Patent Application Publication
(10) Pub. No.: US 2021/0244845 A1
(43) Pub. Date: Aug. 12, 2021

(54) Method Of Processing Amnion To Form A Suture

(71) Applicants: Leslie MARCELIN, Ft. Lauderdale, FL (US); Marie WILLIAMS, Miami, FL (US); Jean HOLEWINSKI, Miami, FL (US)

(72) Inventors: Leslie MARCELIN, Ft. Lauderdale, FL (US); Marie WILLIAMS, Miami, FL (US); Jean HOLEWINSKI, Miami, FL (US)

(21) Appl. No.: 17/172,687

(22) Filed: Feb. 10, 2021

Related U.S. Application Data
(60) Provisional application No. 62/972,448, filed on Feb. 10, 2020.

(51) Int. Cl. A61L 17/08; A61K 35/50; C12N 5/073; A01N 1/02; D04C 1/02; D04C 1/12

(52) U.S. Cl. CPC A61L 17/08; A61K 35/50; C12N 5/0605; A01N 1/0284; D10B 2211/20; D04C 1/12; A61L 2430/40; D10B 2509/04; D04C 1/02

(57) ABSTRACT

A method for processing amniotic membrane to create a suture including cutting dried amnion layer into a designated final strip size, the cut and dried strips are twisted into a thread-like structure, interlacing the thread-like structures and drying the interlaced thread-like structure to form the suture.

### CROSS REFERENCE

The present application for patent claims the benefit of U.S. Provisional Patent Application No. 62/972,448 filed Feb. 10, 2020, and expressly incorporated by reference herein.

### FIELD OF THE INVENTION

The present Invention relates a protocol for the production of amniotic tissue derived products, an amnion membrane, an amnion suture allograft, and a method for the production of amniotic tissue derived products into a biologic suture.

### BACKGROUND OF THE INVENTION

Regenerative wound healing has been extensively studied since 1979 when it was first reported that fetal surgical wounds heal without scarring. Compared to scar-mediated healing in an adult, regenerative healing in a fetus involves rapid reepithelialization, the absence of an inflammatory response, preservation of tissue architecture, and the absence of scar tissue formation.

During gestation, Amnion provides both physical and systemic protection to the fetus. It is an immune-privileged protective barrier with natural anti-inflammatory, anti-scarring and antimicrobial properties. The amnion contains various proteins that support cell proliferation, movement and differentiation.

Amniotic Allografts are tissue grafts derived from placenta for orthopedic surgery, wound repair, and accelerated healing. Regenerative treatments, such as amniotic allografts, have been shown to substantially shorten the time it takes for patients to make a healthy recovery from joint replacement, join reconstruction and spine surgery, returning them to normal activities quicker.

An allograft can be derived from human placental tissues for use as a wound covering in the treatment of localized tissue defects or areas of inflammation. An amniotic tissue graft replaces the connective tissue that forms a protective covering for tendons, the spine, and the like and can reduce the amount of time it takes to recover from procedures. In tendon surgery, one challenge is restoring proper movement to the area because the gliding function provided by the tendon's protective covering or fascia is interrupted by the adhesions left from the surgery. Using amniotic tissue allografts restores that movement.

### SUMMARY OF THE INVENTION

What is needed is an amnion suture allograft. According to one aspect of the invention, this need is met by a processing method for the production of amniotic tissue derived products and to make an amnion suture allograft.

One aspect of the invention is a method for processing amniotic membrane to create a suture. The method of processing amniotic membrane comprises cutting dried amnion layer into a designated final strip size. The cut and dried strips are twisted into a thread-like structure. According to one aspect of the invention at least three thread-like structures are interlaced. According to one aspect of the invention, the interlacing is a braiding.

Disposable materials are utilized to process the amniotic membrane to create a suture. Preferably, the technician or operator utilizes proper personal protective equipment (PPE) and gowning materials including but not limited to scrubs, shoe covers, face masks, face shields, and sterile gowns. Other materials, which may be disposable, include sterilized scalpels and a cutting board with measurements.

Additional materials include sterilized scissors, measuring utensils, sterile rulers, and dura-clamps. According to one aspect of the invention, the dura-clamps have a width of 0.625 in.

### DESCRIPTION OF THE FIGURES

FIG. 1 is a chart of disposable materials used to process the amniotic membrane;

FIG. 2 is a chart of non-disposable materials used to process the amniotic membrane;

FIG. 3 is a chart of equipment used to process the amniotic membrane; and

FIG. 4 is a chart of reagents used to process the amniotic membrane.

### DETAILED DESCRIPTION

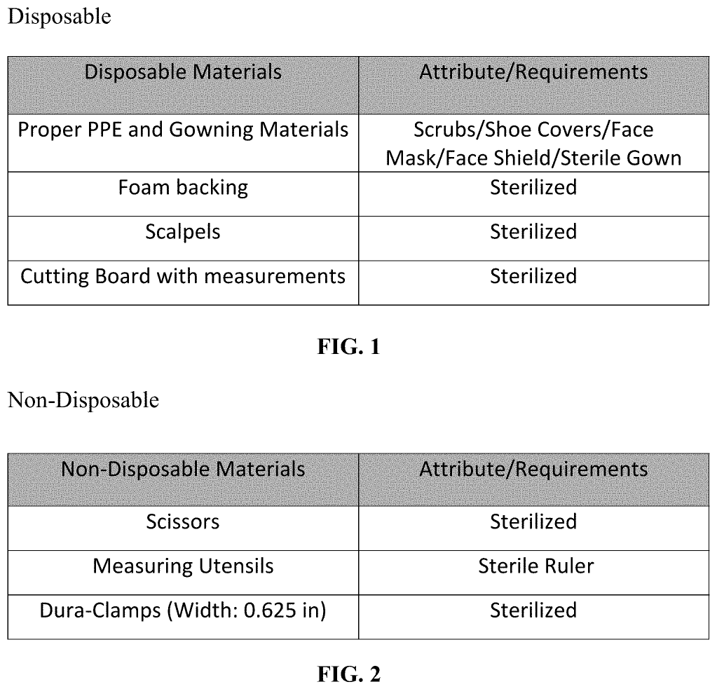

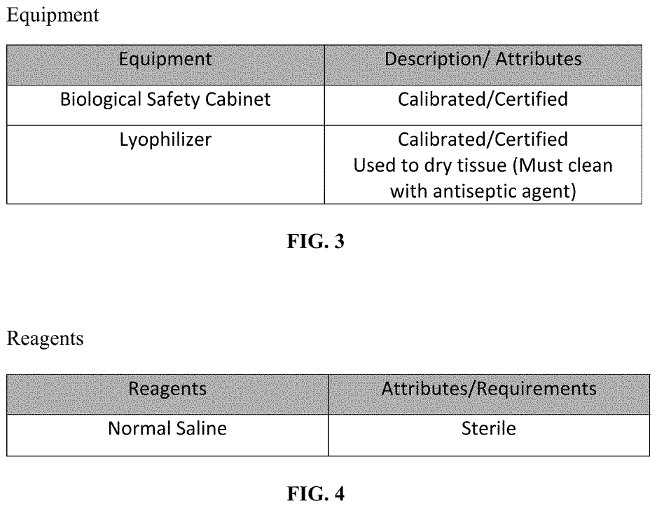

FIG. 1 is a chart of disposable materials used to process the amniotic membrane. FIG. 2 is a chart of non-disposable materials used to process the amniotic membrane. The disposable and nondisposable materials are generally used in the processing of the amniotic membrane to create an amnion suture allograft using the equipment listed in FIG. 3 and the reagents listed in FIG. 4.

The following procedure is used to process amniotic membrane according to one aspect of the invention. Initially, placental tissue (amnion sheets) are harvested. Typically, after the amnion sheets are harvested, they are frozen for storage and transportation. Before processing, the placental tissue is thawed at room temperature for 10-12 hours. After harvesting and/or thawing, the amniotic membrane is cleaned. Saline is well known to replace lost body fluids and salts. Saline makes a great ingredient because it helps keep the tissue naturally, without using harmful detergents. Typically, a saline solution is used for the cleaning, specifically 0.9 sodium chloride is the cleaning agent used in this process.

The cleaning process begins by preparing jars or other suitable container for rinsing. Each jar should be labeled to identify the tissue being cleaned. It should be noted that sterile techniques must be observed during the processing of the tissue.

The placental sack is dissected into its amnion and chorion layers. The goal is to separate the Amnion from the Chorion layer. The Amnion layer is gently scraped to remove debris from the chorion layer. The goal is to remove the Chorion layer due to its high concentration of pro-inflammatory cytokines.

Typically technicians do this using their fingers, although an automated process can be used. After scraping, the tissue is rinsed with saline until it is clear of debris.

After cleaning, the amnion layers are cut into strips. According to one aspect of the invention, the strips are 0.6 cm×60.0 cm. It should be noted that a wide range of sizes can be used, but 0.6 cm×60.0 cm has the best consistency. After the strips are cut, each strip is twisted or wound into a threadlike structure or filament. The diameter of each threadlike structure or filament is about 0.35 mm. It should be noted that the twisting process will reduce the length of each strip. In other words, the threadlike structure or filament is shorter than the strip from which it was formed.

Once wound, the filaments are grouped into groups of three. It should be noted that other groupings are foreseeable including but not limited to four to ten or more filaments. Once grouped, the filaments are placed individually in a sterile saline bath for a set time. According to one aspect of the invention the saline bath is about two seconds. After the sterile bath the filaments are placed on a sterile tray. According to one aspect of the invention the filaments dry for about 20 minutes.

After the filaments are dried, the amnion filaments are lined up in a way in which they are not touching. The group of filaments, typically three filaments, are clamped together. According to one aspect of the invention, the filaments are clamped at one end. The filaments are then interlaced. According to one aspect of the invention the three filaments are interlaced in such a way that they cross one another and are laid together in diagonal formation. This interlacing is a braiding step.

After braiding, each suture, which is a braided group of filaments, is soaked in sterile saline for a set time. According to one aspect of the invention, the time for soaking the suture is 2-3 seconds. After soaking the suture is clamped at an end and the braided suture and gently undergoes a twisting and squeezing operation from end to end. Each strand lightly fuses to the other, which helps to prevent hardening during the process. The twisting and squeezing operation is performed to loosen and maintain a desired length for the suture.

After the twisting and squeezing operation the suture is placed on a foam backing. The tissue and foam backing into lyophilizer to execute a water removal process to preserve the tissue. The drying cycle setting for the lyophilizer is a vacuum cycle. Typically, the cycle is a 30-45 min cycle to dry the product.

Once the suture is dried, the suture is removed from the lyophilizer and prepared for testing and packaging. Testing of the suture includes, but is not limited to the elongation of the suture, a yield point of the suture, and tensile strength of the suture.

In an alternative embodiment, after cleaning the reassembled anion and/or chorion tissue layers as placed on a foam backing into the lyophilizer. As above, the drying cycle setting is a vacuum cycle. A typical vacuum cycle is about a 30-45 minute cycle, repeated until the product is dry. The tissue is removed from the lyophilizer and prepared for future processing. The processing includes cutting the dried tissue into designated final sizes using sterile scalpels. The cut dried tissue is packaged in sterile peel pouches.

According to one aspect of the invention a notch is cut on a lower left corner, or other location, to indicate epithelial or stromal side orientation.

The sutures are stored at room temperature. However, other temperatures can be used. During production, one random unit is selected for destructive testing. Destructive testing includes, but is not limited to measuring the consistency of each batch for characteristics such as tensile strength and aesthetics. Additionally, three random units are selected for quality control testing. The testing includes, but is not limited to, infectious disease analyses, protein analyses, and bacterial analyses. Preferably, serology testing for infectious disease is performed prior to processing so it would not typically be included in the QC testing. According to one aspect of the invention, an ELISA assessment for protein analysis is performed as well as taking cultures before final packaging for bacteria analysis.

The finished sutures undergo e-Beam sterilization. Typically, e-Beam sterilization is performed overnight or for 10-15 minutes. After sterilization, the sutures are packages and stored for distribution and use.

The procedure to prepare the amnion for processing into sutures can be summarized as follows:

Procedure
3.1 Thaw placental tissue at room temperature for 10-12 hours
   3.1.1 Prepare jars for rinsing.
   3.1.2 Label each Jar
   3.1.3 Note, sterile technique must be observed from this point
   3.1.4 Dissect placental sack
      3.1.4.1 Separate amnion and chorion layers
      3.1.4.2 For each layer, gently scrape tissue utilizing technician's fingers or other appropriate tool to remove debris from amnion or chorion tissues. Rinse in NS until clear of debris.
   3.1.5 Reassemble amnion and chorion tissue layers on foam backing
      3.1.5.1 Place membrane on the foam backing (Better handling)
   3.1.6 Place tissue and foam backing into lyophilizer
      3.1.6.1 Drying cycle setting: Vacuum cycle
      3.1.6.2 30-45 min cycles until product is dry
   3.1.7 Remove tissue from Lyophilizer and prep for future processing.
      The above steps are performed prior to cutting the strip to start processing the sutures.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve substantially the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A method for processing amniotic membrane to create a suture, comprising:
harvesting placental tissue as amnion sheets;
cleaning the amnion sheets;
separating the amnion sheets into an amnion layer and a chorion layer;
cleaning the amnion layer to remove debris from the chorion layer;
rinsing the amnion layer until it is clear of debris;
cutting the amnion layer into a plurality of strips;
forming each strip into a threadlike structure;
placing the threadlike structures into a sterile bath;
executing a first drying cycle to dry the threadlike structures;
clamping a plurality threadlike structures at one end;
interlacing the threadlike structures;
performing a twisting and squeezing operation on the interlaced threadlike structures; and
executing a second drying cycle to dry the interlaced threadlike structures yielding the suture.

2. The method for processing amniotic membrane according to claim 1, further comprising:
freezing the amnion sheets after harvesting for at least one of storage and transportation.

3. The method for processing amniotic membrane according to claim 2, further comprising:
thawing the amnion sheets prior to further processing.

4. The method for processing amniotic membrane according to claim 1, wherein a saline solution is used for the cleaning.

5. The method for processing amniotic membrane according to claim 1, wherein each of the plurality of strips is 0.6 cm×60.0 cm.

6. The method for processing amniotic membrane according to claim 1, wherein the threadlike structure is formed by at least one of twisting and winding.

7. The method for processing amniotic membrane according to claim 6, wherein a diameter of each threadlike structure is about 0.35 mm.

8. The method for processing amniotic membrane according to claim 1, wherein the threadlike structures are placed into the sterile bath in groups of at least three.

9. The method for processing amniotic membrane according to claim 1, wherein the first drying cycle is about 20 minutes.

10. The method for processing amniotic membrane according to claim 1, wherein three filaments are interlaced in such a way that they cross one another and are laid together in diagonal formation.

11. The method for processing amniotic membrane according to claim 10, wherein the interlacing is a braiding step.

12. The method for processing amniotic membrane according to claim 1, wherein the twisting and squeezing operation causes respective strands of the threadlike structures to lightly fuse to each other.

13. The method for processing amniotic membrane according to claim 2, further comprising:
placing the interlaced threadlike structures on a foam backing after the twisting and squeezing.

14. The method for processing amniotic membrane according to claim 1, wherein at least one of the first drying cycle and the second drying cycle is performed in a lyophilizer.

15. The method for processing amniotic membrane according to claim 14, wherein the at least one of the first drying cycle and the second drying cycle is a vacuum cycle.

16. The method for processing amniotic membrane according to claim 1, further comprising:
cutting the suture to a desired length after the second drying cycle.

17. The method for processing amniotic membrane according to claim 16, further comprising:
packaging each suture in a respective sterile peel pouch.
